(19) United States
(12) Patent Application Publication
(10) Pub. No.: US 2010/0098772 A1
(43) Pub. Date: Apr. 22, 2010

(54) DRUG DELIVERY SYSTEMS AND METHODS FOR TREATING NEOVASCULARIZATION

(75) Inventors: Michael R. ROBINSON, Irvine, CA (US); Susan Y. Tsai, Costa Mesa, CA (US); Alexandra S. Almazan, Santa Ana, CA (US); Wendy M. Blanda, Tustin, CA (US); Patrick M. Hughes, Aliso Viejo, CA (US); James A. Burke, Santa Ana, CA (US); Scott M. Whitcup, Laguna Hills, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/255,497

(22) Filed: Oct. 21, 2008

(51) Int. Cl. A61K 9/14; A61K 39/395; A61P 27/02; A61K 31/7052

(52) U.S. Cl. 424/501; 424/145.1; 424/158.1; 514/44 R; 514/54

(57) ABSTRACT

Anti-angiogenesis compositions, and methods of using such compositions, useful for intraocular to treat neovascularization. The compositions can have viscosities at about 25° C. of at least about 10 cps or about 100 cps at a shear rate of 0.1/second. In a preferred embodiment, the viscosity at 25° C. is in the range of from about 80,000 cps to about 300,000 cps.

### BACKGROUND

The present invention relates to drug delivery systems and methods for treating an anterior ocular condition. In particular, the present invention relates to biodegradable, sustained release drug delivery systems and methods for treating anterior segment ocular (i.e. corneal) neovascularization. The drug delivery system can comprise an anti-neovascular agent (such as an anti-VEGF agent) and be a solid or liquid (i.e. a gel, suspension or emulsion) drug delivery system.

The exterior surface of the normal globe mammalian eye has a layer of tissue known as conjunctival epithelium, under which is a layer of tissue called Tenon's fascia (also called conjunctival stroma). The extent of the Tenon's fascia extending backwards across the globe forms a fascial sheath known as Tenon's capsule. Under Tenon's fascia is the episclera. Collectively, the conjunctival epithelium and the Tenon's fascia is referred to as the conjunctiva. As noted, under Tenon's fascia is the episclera, underneath which lies the sclera, followed by the choroid. Most of the lymphatic vessels and their associated drainage system, which is very efficient at removing therapeutic agents placed in their vicinity, are present in the conjunctiva of the eye.

An ocular condition can be characterized by angiogenesis, which is by formation of new blood vessels. The infiltrative growth of new blood vessels can disrupt or destroy ocular tissue; thus the inhibition of angiogenesis can also be considered to provide protection to affected eye cells, such as retinal neurons or corneal cells. Anterior ocular conditions characterized by angiogenesis include corneal neovascularization.

Corneal neovascularization is the excessive in growth of blood vessels from the limbus into adjacent corneal tissues, probably due to a low level of oxygen in the tissues so invaded. The new blood vessels can extend into superficial and deep corneal stroma. Corneal neovacularization can develop into anterior ocular inflammation, trachoma, viral interstitial keratitis or microbial keratoconjunctivitis. Corneal neovascularization can be caused by wearing contact lens and corneal neovascularization can be associated with corneal scarring and vision loss.

Vascular epithelial growth factor (“VEGF”) is a family of proteins involved in angiogenesis, that is the growth of blood vessels from pre-existing vasculature. VEGF also enhances vascular permeability. Anti-VEGF agents, which inhibit either VEGF itself or the VEGF receptor present in the eye in order to thereby prevent angiogenesis, include monoclonal antibodies such as ranibizumab (LUCENTIS®; rhuFab V2) and bevacizumab (AVASTIN®; rhuMab-VEGF), nucleic acids (aptamers such as MACUGEN®, (pegaptanib) a PEGylated RNA aptamer, and siRNAs directed to VEGF RNA). Bevacizumab is a full-length anti-VEGF antibody approved for use in metastatic colon cancer. Ranibizumab is a humanized anti-VEGF monoclonal antibody fragment that inhibits all isotypes of VEGF and pegaptanib is a VEGF-neutralizing aptamer that specifically inhibits one isoform of VEGF (VEGF-165).

Aqueous solution of bevacizumab has been administered subconjunctival and sub- Tenon to treat corneal neovascularization but with the effect lasting only a couple of weeks after administration.

A hydrogel is a colloidal gel formed as a dispersion in water or other aqueous medium. Thus a hydrogel is formed upon formation of a colloid in which a dispersed phase (the colloid) has combined with a continuous phase (i.e. water) to produce a viscous jellylike product; for example, coagulated silicic acid. A hydrogel is a three-dimensional network of hydrophilic polymer chains that are crosslinked through either chemical or physical bonding. Because of the hydrophilic nature of the polymer chains, hydrogels absorb water and swell. The swelling process is the same as the dissolution of non-crosslinked hydrophilic polymers. By definition, water constitutes at least 10% of the total weight (or volume) of a hydrogel.

Examples of hydrogels include synthetic polymers such as polyhydroxy ethyl methacrylate, and chemically or physically crosslinked polyvinyl alcohol, polyacrylamide, poly(N-vinyl pyrrolidone), polyethylene oxide, and hydrolyzed polyacrylonitrile. Examples of hydrogels which are organic polymers include covalent or ionically crosslinked polysaccharide-based hydrogels such as the polyvalent metal salts of alginate, pectin, carboxymethyl cellulose, heparin, hyaluronate and hydrogels from chitin, chitosan, pullulan, gellan and xanthan. The particular hydrogels used in our experiment were a cellulose compound (i.e. hydroxypropylmethylcellulose [HPMC]) and a high molecular weight hyaluronic acid (HA).

Hyaluronic acid is a polysaccharide made by various body tissues. U.S. Pat. No. 5,166,331 discusses purification of different fractions of hyaluronic acid for use as a substitute for intraocular fluids and as a topical ophthalmic drug carrier. Other U.S. patent applications which discuss ocular uses of hyaluronic acid include Ser. Nos. 11/859,627; 11/952,927;10/966,764; 11/741,366; and 11/039,192

Formulations of macromolecules for intraocular use are known, See eg U.S. patent applications Ser. Nos. 11/370,301; 11/364,687; 60/721,600; 11/116,698 and 60/567,423; 11/695,527. Use of various active agents is a high viscosity hyaluronic acid is known. See eg U.S. patent applications Ser. Nos. 10/966,764; 11/091,977; 11/354,415; 60/519,237; 60/530,062, and; 11/695,527.

It is known to administer a drug depot to the posterior (i.e. near the macula) sub-Tenon space. See eg column 4 of U.S. Pat. No. 6,413,245. Additionally, it is known to administer a polylactide implant to the sub-tenon space or to a suprachoroidal location. See eg published U.S. Pat. No. 5,264,188 and published U.S. patent application 20050244463

An intraocular drug delivery system can be made of a biodegradable polymeric such as a poly(lactide) (PLA) polymers, poly(lactide-co-glycolide) (PLGA) polymers, as well as copolymers of PLA and PLGA polymers. PLA and PLGA polymers degrade by hydrolysis, and the degradation products, lactic acid and glycolic acid, are metabolized into carbon dioxide and water. Polylactide (PLA) polymers exist in 2 chemical forms, poly(L-lactide) and poly(D,L-lactide). The pure poly(L-lactide) is regioregular and therefore is also highly crystalline, therefore degrades in vivo at a very slow rate. The poly(D,L-lactide) is regiorandom which leads to more rapid degradation in vivo. Therefore a PLA polymer which is a mixture of predominantly poly(L-lactide) polymer, the remainder being a poly(D-lactide) polymer will degrade in vivo at a rate slower that a PLA polymer which is predominantly poly(D-lactide) polymer. A PLGA is a co-polymer that combines poly(D,L-lactide) with poly(glycolide) in various possible ratios. The higher the glycolide content in a PLGA the faster the polymer degradation.

Drug delivery systems have been formulated with various active agents. For example, it is known to make PLGA and PLA implants (as rods, wafers, discs, and filaments), intended for intraocular use by a melt extrusion methods. See eg published U.S. patent application 20050244471, and U.S. patent application Ser. No. 10/918,597. Additionally, it is known to make brimonidine poly lactic acid polymer implants and microspheres intended for intraocular use. See eg published U.S. patent applications 20050244463 and 20050244506, and U.S. patent application Ser. No. 11/395,019. Furthermore, it is known to make bimatoprost containing polylactic acid polymer implants and microspheres intended for intraocular use. See eg published U.S. patent applications 2005 0244464 and 2006 0182781, and U.S. patent applications Ser. Nos. 11/303,462, and; 11/371,118.

EP 488 401 discusses intraocular implants, made of certain polylactic acids, to be applied to the interior of the eye after a surgical operation for disorders of the retina/vitreous body or for glaucoma. EP 430539 discusses use of a bioerodible implant which is inserted in the suprachoroid.

U.S. application Ser. No. 11/565,917 filed Dec. 1, 2006 discloses intraocular (including sub-tenon's) administration of various solid, drug-containing implants.

U.S. patent applications Ser. Nos. 11/742,350; 11/859,310; 11/952,938; 11/364,687 discuss use of intraocular compositions comprising anti-VEGF therapeutic agent, such as bevacizumab. Formulations of macromolecules for intraocular use are known, See eg applications Ser. Nos. 11/370,301; 11/364,687; 60/721,600; 11/116,698 and 60/567,423.

The anti-neovascular agent bevacizumab has been administered subconjunctival and sub-Tenon's to treat corneal neovascularization. The bevacizumab was so administered in aqueous solution, that is as a non-sustained release formulation and the reduction in neovascularization lasted only for 2 to 3 weeks. What is needed therefore is a sustained-release formulation (capable of releasing the active agent over 1-6 months) to thereby effectively treat corneal neovascularization.

### SUMMARY

The present invention meets this need by providing a sustained-release formulation (capable of releasing the active agent over 1-6 months) to thereby effectively treat corneal neovascularization. We determined that a basal level of vascular endothelial growth factor (VEGF) is required for maintenance of new vessel growth and that our sustained-release anti-VEGF compound drug delivery system can reduce the basal VEGF levels below the threshold required for new vessel stability and the endothelial cells would undergo apoptosis. Our invention can reduce abnormal vessels in the cornea thereby reducing pannus formation to improve the clarity of the cornea and improve visual acuity.

Definitions

As used herein, the words or terms set forth below have the following definitions.

“About” means approximately or nearly and in the context of a numerical value or range set forth herein means ±10% of the numerical value or range recited or claimed.

“Anti-neovascular agent” means a compound which has an anti-angiogenic effect when administered to an eye such as by intravitreal injection or implantation.

“Anti-VEGF agent” means a compound which inhibits an activity or an effect of VEGF, and includes bevacizumab, ranibizumab, pegaptanib, VEGF-neutralizing aptamers, anti-VEGF monoclonal antibodies, siRNAs, corticosteroids such as anacortave acetate, triamcinolone acetonide and fluocinolone acetonide; receptor tyrosine kinase inhibitors, such as vatalanib and Ruboxistaurin, squalamine lactate, and; growth factors, including pigment epithelium-derived factor.

“Biocompatible” with regard to a drug delivery system means that upon intraocular administration of the drug delivery system to a mammalian eye a significant immunogenic reaction does not occur.

“Bioerodible polymer” means a polymer which degrades in vivo. The polymer can be a gel or hydrogel type polymer, PLA or PLGA polymer or mixtures or derivatives thereof. The words “bioerodible” and “biodegradable” are synonymous and are used interchangeably herein.

“Drug delivery system” means a liquid, gel, hydrogel, high viscosity formulation, solid implant or microspheres from which a therapeutic amount of a therapeutic agent can be released upon in vivo administration of the drug delivery system, without any requirement that the drug delivery system by sutured to ocular tissue or otherwise fixed in place by an attachment means.

“Entirely free (i.e. “consisting of” terminology) means that within the detection range of the instrument or process being used or referenced, the substance cannot be detected or its presence cannot be conclusively confirmed.

“Essentially free” means that only trace amounts of other substances, or a reference substance (such trace amounts not having a substantial effect in the application), can be detected.

“Intraocular” means within or under an ocular tissue. An Intraocular administration of a drug delivery system includes administration of the drug delivery system to a sub-Tenon, subconjunctival, suprachoroidal, intravitreal and like locations. An Intraocular administration of a drug delivery system excludes administration of the drug delivery system to a topical, systemic, intramuscular, subcutaneous, intraperitoneal, and the like location.

“Ocular condition” means a disease, aliment or condition which affects or involves the eye or one of the parts or regions of the eye. The eye includes the eyeball and the tissues and fluids which constitute the eyeball, the periocular muscles (such as the oblique and rectus muscles) and the portion of the optic nerve which is within or adjacent to the eyeball.

A front of the eye (or “anterior” or “anterior segment”) ocular condition is a disease, ailment or condition which affects or which involves an ocular region or site, such as a periocular muscle, an eye lid or an eye ball tissue or fluid which is located anterior to the posterior wall of the lens capsule or ciliary muscles. Thus, a front of the eye ocular condition primarily affects or involves, the conjunctiva, the cornea, the conjunctiva, the anterior chamber, the iris, the posterior chamber (behind the iris but in front of the posterior wall of the lens capsule), the lens and the lens capsule as well as blood vessels, lymphatics and nerves which vascularize, maintain or innervate an anterior ocular region or site. A front of the eye ocular condition includes a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; corneal neovascularization; refractive disorders and strabismus. Glaucoma can be considered to be a front of the eye ocular condition because a clinical goal of glaucoma treatment can be to reduce a hypertension of aqueous fluid in the anterior chamber of the eye (i.e. reduce intraocular pressure).

A posterior (or back of the eye) ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition, such as for example, macular degeneration (such as non-exudative age related macular degeneration and exudative age related macular degeneration); choroidal or retinal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can also be considered a posterior ocular condition because a therapeutic goal of glaucoma treatment is to prevent the loss of or reduce the occurrence of loss of vision due to damage to or loss of retinal cells or optic nerve cells (i.e. neuroprotection).

“Pharmaceutical composition (synonymously a “composition”) means a formulation which contains at least one active ingredient (for example an anti-neovascular agent) and a carrier for the active agent. “Formulation” means that there is at least one additional ingredient in the pharmaceutical composition besides the active ingredient. A pharmaceutical composition is therefore a formulation which is suitable for diagnostic or therapeutic administration (e.g., by intraocular injection or by insertion of a depot or implant) to a subject, such as a human patient. A pharmaceutical composition can include one or more excipients, buffers, carriers, stabilizers, preservatives and/or bulking agents, and is suitable for administration to a patient to achieve a desired effect or result. The pharmaceutical compositions disclosed herein can have diagnostic, therapeutic, cosmetic and/or research utility in various species, such as for example in human patients or subjects.

“Suitable for insertion (or implantation) in (or into) an ocular region or site” with regard to a drug delivery system, means a drug delivery system which has a size (dimensions) such that it can be administered, injected, inserted or implanted without causing excessive tissue damage and without unduly physically interfering with the existing vision of the patient into which the implant is implanted or inserted.

“Sustained” as in “sustained period” or “sustained release” means for a period of time greater than ten days, preferably for at least 20 days (i.e. for a period of time from 20 days to 365 days), and most preferably for at least 30 days. A sustained release can persist for between one month and about six months.

Viscosity values herein mean the viscosity at 25° C., unless specifically indicated otherwise.

Our invention encompasses compositions for administration (as by injection) into an anterior ocular location. The compositions comprise an anti-neovascular agent present in a therapeutically effective amount.

Whether nucleic acid or polypeptide in nature use of anti-neovascular therapeutic agents in a sustained release drug delivery system present specific challenges. systems. The drug formulation must above all be substantially non-toxic to intraocular tissues. When such a formulation comprises a liquid carrier, it is very advantageous for the carrier component to possess a refractive index that is substantially similar to that of the aqueous humor or the vitreous humor (depending upon in which chamber the formulation is introduced), so that the patient's vision is not substantially adversely affected, such as by changes in focus, following administration, for example injection, of the therapeutic composition into an intraocular tissue. Formulations having a refractive index of water (approximately 1.33, depending on the wavelength of light), for example, could create enough of a difference in refractive index at the boundary of injected formulation and the vitreous humor following injection to adversely affect vision in the patient during a time following administration.

Additionally, given the complex folding necessary to give proteins their biological activity, it is surprising that a solution comprising relatively high concentrations of a given viscosity enhancing component, such as 2% hyaluronic acid, at an given pH, such as between about 6.5 to about 8.0, would permit anti-neovascular anti-neovascular agents, such as proteins or polypeptides, to retain a biologically active conformation without denaturation. As opposed to “small” molecules, which either lack a tertiary structure or are less dependent for their activity on their three dimensional conformation, proteins are capable of being denatured by any of a variety of changes in their environment, including heat, cold, high salt concentrations, the presence of chaotropes(agents that cause molecular structure to be disrupted; in particular, those structures formed by nonbonding forces such as hydrogen bonding, Van der Waals interactions, and the hydrophobic effect).

Similarly, certain nucleic acids, require the maintenance of a given three dimensional conformation in order to retain their desired anti-neovascular agent activity. This is particularly true of certain nucleic acid aptamers, which rely on a biological activity, such as a enzymatic or receptor inhibitory activity for their activity. This is also true of enzymatic nucleic acids such as ribozymes. Again, it is surprising that high concentrations of a viscosity enhancing component in a drug formulation would not lead to loss of this activity through unfolding and denaturation of the nucleic acids' tertiary structure.

In certain embodiments the formulation of the present invention may comprise a suspension of particles or crystals comprising the therapeutic component or of biodegradable polymers within which or on the surface of which a population of the therapeutic component is deposited or incorporated. For example, the particles may comprise a biodegradable microparticle, such as a microsphere or nanosphere, and are capable of being injected or surgically placed within the anterior or posterior segment of the mammalian eye.

In a preferred embodiment the anti-neovascular agent is insoluble and forms a suspension of particles or crystals. In the case of very water-soluble anti-neovascular agents such as oligonucleotides, charge complexation can be used to create such particles. For example, polycations such as polylysine or protamine can be used to form insoluble complexes with polyanions such as oligonucleotides. Anti-neovascular drugs in suspension are more likely to remain chemically stable during long-term storage than in aqueous solution.

In one embodiment an intraocular drug delivery formulation comprises a therapeutic component comprising a non-neurotoxic macromolecule therapeutic agent and a viscosity inducing component. In certain embodiments the formulation may also contain a polymeric component associated with the therapeutic component to permit the therapeutic component to be released into the interior of an eye of an individual for at least about one week after the drug delivery system is placed in the eye.

In accordance with the present invention, the therapeutic agent of the present systems can comprise, consist essentially of, or consist entirely of an anti-neovascular In particularly preferred embodiments the anti-neovascular agent is an anti-VEGF agent, such as a short interfering ribonucleic acids (siRNAs), oligonucleotide aptamers, ranibizumab (sold under the name LUCENTIS®), bevacizumab (sold under the name AVASTIN®), pegaptanib, such as MACUGEN®, (VEGF or VEGFR inhibitors), rapamycin, and cyclosporine.

The polymeric hyaluronic acid (“HA”) of the present compositions is present in an amount effective to increase the viscosity of the composition. The polymeric hyaluronic acid can be a polymeric sodium hyaluronate. The HA is substantially clear in solution, and present in an amount such that the refractive index of the resulting anti-neovascular agent-containing composition is substantially similar to that of the cornea in order to prevent deleterious changes in vision after administration (such as intraocular delivery) of the composition to a patient.

In one embodiment, the composition has a viscosity of at least about 10 cps or at least about 100 cps, preferably at least about 1,000 cps, more preferably at least about 10,000 cps and still more preferably at least about 70,000 cps, for example, up to about 250,000 cps, or about 300,000 cps, at a shear rate of 0.1/second at about 25° C. Preferably, the present compositions are structured or formulated to be effectively, for example, manually, injected into a posterior segment of an eye of a human or animal, preferably through a 27 gauge needle, more preferably through a 29 or 30 gauge needle.

Without wishing to limit the invention to any particular theory of operation, it is believed that the use of relatively high viscosity compositions, as described herein, provides for effective, and preferably substantially long-lasting delivery of the anti-neovascular agent while, at the same time, being injectable into the anterior segment of an eye through conventionally, or even smaller than conventionally, used needles. In embodiments in which the anti-neovascular agent is delivered in part as marginally or slowly soluble particles, the HA s also effective to aid in keeping the particles in suspension, rather than being largely or mostly simply deposited on the bottom surface of the posterior segment of the eye.

In one embodiment of the invention, the anti-neovascular agent is present in a plurality of particles which are substantially uniformly suspended in the composition and remain substantially uniformly suspended in the composition for at least about 1 week, preferably at least about 2 weeks or at least about 1 month, and still more preferably at least about 6 months or at least about 1 year or at least about 2 years, without requiring resuspension processing, that is, without requiring being shaken or otherwise agitated to maintain the anti-neovascular agent particles substantially uniformly suspended in the composition.

Compositions having such substantially uniform suspension of anti-neovascular agent particles, so as to be able to provide a consistent and accurate dose upon administration to an eye, provide substantial advantages relative to the prior art. In particular, the present compositions may be manufactured, shipped and stored for substantial periods of time without the anti-neovascular agent particles precipitating from the remainder of the composition. Having the anti-neovascular agent particles maintained substantially uniformly suspended in the composition allows the composition to provide long term dosing consistency and accuracy per unit dose amount administered, without any need to resuspend the anti-neovascular agent particles.

The composition can have a viscosity of at least about 10 cps at a shear rate of about 0.1/second at 25 degrees C. and is injectable into the vitreous of a human eye, for example through a 27 gauge needle. By reducing the viscosity of our formulation it can be injected into the vitreous through a 28, 29, or 30 gauge needle.

A detailed embodiment within the scope of our invention is a pharmaceutical composition for treating an anterior ocular condition, comprising a anti-neovascular agent; polymeric hyaluronate (in which the anti-neovascular agent is present); sodium chloride; sodium phosphate, and water. “Hyaluronate” is used synonymously with “hyaluronic acid”. The pharmaceutical composition can have a viscosity at a shear rate of about 0.1/second of between about 80,000 cps to about 300,000 cps, preferably from about 100,000 cps to about 300,000 cps, and most preferably from about 180,000 cps to about 225,000 cps. Note that the pharmaceutical composition can have a viscosity at a shear rate of about 0.1/second of between about 80,000 cps and about 300,000 cps, and that when the pharmaceutical composition has a viscosity at a shear rate of about 0.1/second of between about 100,000 cps and about 150,000 cps it can be injected into the vitreous through a 27, 28, 29, or 30 gauge needle. Even with a 300,000 cps it is believed the present formulations can be injected through a 30 gauge needle due to shear thinning once the formulation is in movement in the syringe. The sodium phosphate present in the pharmaceutical composition can comprise both monobasic sodium phosphate and dibasic sodium phosphate. Additionally, the pharmaceutical composition can comprise an effective dose of a anti-neovascular agent, between about 2% w/v hyaluronate and about 3% w/v hyaluronate, about 0.6% w/v sodium chloride and between about 0.03% w/v sodium phosphate and about 0.04% w/v sodium phosphate. Alternately, the pharmaceutical composition can comprise between about 0.5% w/v hyaluronate and about 6% w/v hyaluronate. If desired the hyaluronate can be heated to decrease its molecular weight (and therefore its viscosity) in the formulation.

The pharmaceutical composition can also comprises between about 0.6% w/v sodium chloride to about 0.9% w/v sodium chloride. Generally, more sodium chloride is used in the formulation as less phosphate is used in the formulation, for example 0.9% sodium chloride can be used if no phosphate is present in the formulation, as in this manner the tonicity of the formulation can be adjusted to obtain the desired isotonicity with physiological fluid. The pharmaceutical composition can comprise between about 0.0% w/v sodium phosphate and 0.1% w/v sodium phosphate. As noted, more phosphate can be used in the formulation if less sodium chloride is present in the formulation so as to obtain a desired pH 7.4 buffering effect.

A pharmaceutical composition within the scope of our invention for treating an anterior ocular condition can, in certain embodiments, comprise a anti-neovascular agent present in a therapeutically effective amount as a plurality of particles, a HA in an amount effective to increase the viscosity of the composition, and an aqueous carrier component, wherein the composition has a viscosity of at least about 10 cps at a shear rate of 0.1/second at 25 degrees C. and is injectable intra corneal and wherein the pharmaceutical composition releases the anti-neovascular agent slowly over a period of up to at least about 45 days after the intra corneal injection.

Our invention encompasses a method for treating an anterior ocular condition, the method comprising the step of sub-tenon administration of a sustained release pharmaceutical composition implant comprising a anti-neovascular agent present in a therapeutically effective amount, a polymeric hyaluronic acid in an amount effective to increase the viscosity of the composition, and an aqueous carrier component, wherein the composition has a viscosity of at least about 10 cps at a shear rate of 0.1/second and is injectable into the vitreous of a human eye, and wherein the posterior ocular condition is treated for up to about 30 weeks by the anti-neovascular agent of the present formulation. In this method the pharmaceutical composition can comprise an anti-neovascular agent, polymeric hyaluronate, sodium chloride, sodium phosphate, and water. Additionally, the administration can be injected through a 27 gauge needle into the cornea of a human eye.

Our invention also includes, when the anti-neovascular agent is not entirely soluble in the aqueous carrier, a process for making a pharmaceutical composition by (a) mixing particles of the anti-neovascular agent with sodium chloride crystals, and about 35% to about 40% of the total volume of the water (water for injection) used to make the formulation; (b) heating the anti-neovascular agent and sodium chloride mixture to a temperature between about 20° C. and about 35° C., thereby preparing a first part; (c) mixing a sodium phosphate and water, thereby preparing a second part; (d) dissolving sodium hyaluronate with a molecular weight between about 1.0 million Daltons and about 1.9 million Daltons in another about 35% to about 40% of the total water volume used to make the formulation, followed by sterile filtration after the dissolving; (e) lyophilization of the dissolved sodium hyaluronate; (f) reconstitution of the lyophilized, sterile sodium hyaluronate, thereby preparing a third part; and; (g) aseptically combining the first, second and third parts, thereby making a sterile, uniform anti-neovascular agent pharmaceutical composition which is, an opaque white gel suspension suitable for intravitreal injection to treat an ocular condition. Water is added as needed (q.s.) to make the desired gel suspension which is about 80% to about 90% by weight water.

Our invention encompasses a pharmaceutical composition for treating ocular neovascularization, the composition comprising an anti-neovascular agent, and a polymeric hyaluronic acid (or other polysaccharide or polyelectrolyte or protein-based polymer) associated with the anti-neovascular agent, wherein the polymeric hyaluronic acid is present in the composition at a concentration between about 1 mg/ml and about 40 mg/ml, such as between about 10 mg/ml and about 40 to 60 mg/ml, between about 10 mg/ml and about 30 mg/ml and between about 20 mg/ml and about 30 mg/ml. The polymeric hyaluronic acid can comprise from about 1 weight % to about 50 weight % cross-linked polymeric hyaluronic acid, such as from about 1 weight % to about 10 weight % cross-linked polymeric hyaluronic acid. Additionally, the cross-linked, polymeric hyaluronic acid can be made from non-cross linked polymeric hyaluronic acid which has a molecular weight between about 200 kDa and about 2,000 kDa, and the polymeric hyaluronic acid can have a storage modulus (G′) of between about 200 and 400 at 5 Hz at 25° C.

Preferably, the antineovascular agent used in the composition is an anti-VEGF agent, such as ranibizumab, bevacizumab and pegaptanib and derivatives, esters, salts and mixtures thereof. The composition can further comprise biodegradable, polymeric microspheres and the microspheres can incorporate at least some of the anti-neovascular agent.

A detained embodiment of a composition within the scope of our invention for treating ocular neovascularization can comprise bevacizumab, and a polymeric hyaluronic acid associated with the anti-neovascular agent, wherein the polymeric hyaluronic acid is present in the composition at a concentration between about 10 mg/ml and about 30 mg/ml, and the polymeric hyaluronic acid comprises from about 1 weight % to about 10 weight % cross-linked polymeric hyaluronic acid.

Our invention also encompasses a method for treating ocular neovascularization (such as corneal neovascularization) by administering to the eye of patient exhibiting ocular neovascularization a therapeutic amount of a composition comprising an anti-neovascular agent (such as bevacizumab), and a polymeric hyaluronic acid associated with the anti-neovascular agent, wherein the polymeric hyaluronic acid is present in the composition at a concentration between about 10 mg/ml and about 30 mg/ml.

Finally, our invention also encompasses a process for making a composition for treating corneal neovascularization, the composition comprising an anti-neovascular agent, and a polymeric hyaluronic acid associated with the anti-neovascular agent, wherein the polymeric hyaluronic acid is present in the composition at a concentration between about 10 mg/ml and about 30 mg/ml, the process comprising the steps of; (a) solubilize and stabilize the neovascularization agent in solution; (b) lyophilize the solution to obtain a dry powder cake; (c) mix together the powder and the hyaluronic acid polymer, and; (d) centrifuge the mixture at no less than 2500 RPM for about 5 to 10 minutes to remove air from the mixture.

### DRAWINGS

The patent or application file contains at least one drawing executed in color. Copies of this patent application publication with color drawing(s) will be provided by the Office upon request and payment of the necessary fee.

FIG. 1 is a color photograph of the rabbit eye in Example 1 after sub-tenon injection of blue Alexa dye. The arrows in FIG. 1 point to intra-scleral lymphatic vessels which can be seen containing and carrying away the blue dye.

FIG. 2A is an MRI scan showing cross-linked hyaluronic acid (HA) in the sub-Tenon's space 55 minutes following an injection in the eye of the rat scanned in FIG. 2A, as set forth in Example 2.

FIG. 2B is an MRI scan of the same rat in FIG. 2A taken three months after the FIG. 2A scan.

FIG. 2C is a photograph of the rat eye scanned in FIG. 2B. The FIG. 2C photograph was also taken three months after the FIG. 2A injection and scan.

FIG. 3A is a photograph of the Example 3 rabbit eye immediately following a sub-Tenon's injection of microspheres in a cross-linked HA.

FIG. 3B is a photograph of the FIG. 3A rabbit eye 1 month after the sub-tenon injection.

### DESCRIPTION

Our invention is based on the discovery that a sustained release drug delivery system comprising an anti-neovascular agent and a particular high molecular weight carrier (such as a mixture of a non-cross linked polymeric hyaluronic acid and a cross linked polymeric hyaluronic acid) for the anti-neovascular agent can be use to treat anterior neovascularization, such as corneal neovascularization.

Our invention requires an understanding of ocular morphology and structure. The exterior surface of the globe mammalian eye can have a layer of tissue known as Tenon's capsule, underneath which lies the sclera, followed by the choroid. Between Tenon's capsule and the sclera is a virtual space known as a sub-Tenon space. Another virtual space lies between the sclera and the choroid, referred to as the suprachoroidal space. Delivery of a therapeutic agent to an ocular location the front of the eye (such as the ciliary body) can be facilitated by placement of a suitably configured drug delivery system to a location such as the anterior sub-Tenon space, the anterior suprachoroidal space. Additionally, a drug delivery system can be administered within the sclera, for example to an anterior intrascleral location. Upon lateral movement of the therapeutic agent from such drug delivery implant locations it can diffuse or be transported through the conjunctiva and sclera to the cornea. Upon perpendicular movement of the therapeutic agent through the sclera and/or the choroid it can be delivered to anterior structures of the eye. For example, an aqueous humor suppressant for the treatment of ocular hypertension or glaucoma, can be delivered from drug delivery systems placed in the anterior sub-Tenon space, the suprachoroidal space or intrascleral to the region of the ciliary body.

As can be understood an intrascleral administration of a drug delivery system does not place the drug delivery system as close to the vitreous as does a suprachoroidal (between the sclera and the choroid) administration. For that reason an intrascleral administration of a drug delivery system can be preferred over a suprachoroidal administration so as to reduce the possibility of inadvertently accessing the vitreous upon administration of the drug delivery system.

Additionally, since the lymphatic network resides in or above the tenon's fascia of the eye and deeper ocular tissues have a reduced blood flow velocity, administration of a drug delivery system in a sub-tenon and more eye interior location can provide the dual advantages of avoiding the rapid removal of the therapeutic agent by the ocular lymphatic system (reduced lymphatic drainage) and the presence of only a low circulatory removal of the therapeutic agent from the administration site. Both factors favor passage of effective amounts of the therapeutic agent to the ciliary body and trabecular meshwork target tissue.

An important characteristic of a drug delivery system within the scope of our invention is that it can be implanted or injected into an intraocular location (such as an anterior sub-Tenon, subconjunctival or suprachoroidal location) to provide sustained release of a therapeutic agent without the occurrence of or the persistence of significant immunogenicity at and adjacent to the site of the intraocular implantation or injection.

In one embodiment of our invention, a drug delivery system for intraocular administration (i.e. by implantation in the sub-Tenon space) comprises configured, consists of, or consists essentially of at least a 75 weight percent of a PLA and no more than about a 25 weight percent of a poly(D,L-lactide-co-glycolide) polymer.

Within the scope of our invention are suspensions of microspheres which can be administered to an intraocular location through a syringe needle. Administration of such a suspension requires that the viscosity of the microsphere suspension at 20° C. be less than about 300,000 cP. The viscosity of water at 20° C is 1.002 cP (cP is centipoise, a measure of viscosity). The viscosity of olive oil is 84 cP, of castor oil 986 P and of glycerol 1490 cP

In particular embodiments of our invention, the anti-neovascular agent can be an anti-VEGF agent, that is an agent that blocks or reduces the expression of VEGF receptors (VEGFR).

The therapeutic active agent present in our drug delivery systems can be homogeneously dispersed in the biodegradable polymer of the drug delivery system. The selection of the biodegradable polymer used can vary with the desired release kinetics, patient tolerance, the nature of the disease to be treated, and the like. Polymer characteristics that are considered include, but are not limited to, the biocompatibility and biodegradability at the site of implantation, compatibility with the active agent of interest, and processing temperatures. The biodegradable polymer matrix usually comprises at least about 10, at least about 20, at least about 30, at least about 40, at least about 50, at least about 60, at least about 70, at least about 80, or at least about 90 weight percent of the implant. In one variation, the biodegradable polymer matrix comprises about 40% to 50% by weight of the drug delivery system.

Biodegradable polymers which can be used include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers can be crosslinked or non-crosslinked.

Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In certain variations, 25/75 PLGA and/or 50/50 PLGA copolymers are used. In other variations, PLGA copolymers are used in conjunction with polylactide polymers.

Other agents may be employed in a drug delivery system formulation for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The drug delivery systems of our invention can be injected to an intraocular location by syringe or can be inserted (implanted) into the eye by a variety of methods, including placement by forceps, by trocar, or by other types of applicators, after making an incision in the sclera. In some instances, a trocar or applicator may be used without creating an incision. In a preferred variation, a hand held applicator is used to insert one or more biodegradable implants into the eye. The hand held applicator typically comprises an 18-30 GA stainless steel needle, a lever, an actuator, and a plunger. Suitable devices for inserting an implant or implants into a posterior ocular region or site includes those disclosed in U.S. patent application Ser. No. 10/666,872.

The method of administration generally first involves accessing the target area within the ocular region with the needle, trocar or implantation device. Once within the target area, e.g., the vitreous cavity, a lever on a hand held device can be depressed to cause an actuator to drive a plunger forward. As the plunger moves forward, it can push the implant or implants into the target area (i.e. the vitreous).

Various techniques may be employed to make implants within the scope of the present invention. Useful techniques include phase separation methods, interfacial methods, extrusion methods, compression methods, molding methods, injection molding methods, heat press methods and the like.

An embodiment of our invention comprises an anti-VEGF compound, such as a monoclonal antibody (i.e. bevacizumab) formulated in a cross-linked hyaluronic acid (HA). The formulation can be injected in the sub-Tenon's space. The cross-linked hyaluronic acid polymer acts as a reservoir for the monoclonal antibody and is present in the sub-Tenon's area for a number of months. Cross-linked HA demonstrates resistance to the robust clearance mechanisms in the sub-Tenon's space. This characteristic allows for increased residency time of the polymeric HA in the sub-Tenon's space to last for a number of months. The hyaluronic acid use in our formulations preferably has the following preferred characteristics. These characteristics provide a tunable gel formulation in terms of both release kinetics of the anti-VEGF compounds and acceptable rheological/flow properties of the final formulation.
1. high rheological strength (G′>300 at 5 Hz at linear viscoelastic regime)
2. hyaluronic acid concentration between 20 to 40 mg/ml allowing a tunable average pore size
3. degree of crosslinking between 1-8% (w/w)
4. the percent cross-linked HA ranging from 15 to >85%
5. the raw material HA molecular weight between 600-1,500 kDa.
6. crosslinked HA product with soluble HA component having an average molecular weight >400 kDa

Suitable cross-linked, polymeric hyaluronic acids which can serve as a vehicle for an anti-neovascular agent (i.e. by keeping the antineovascular agent aggregated in vivo include the hyaluronic acids old under the trade names Juvederm Ultra Plus, Juvederm30, CaptiqueStar-600 and Voluma by Allergan, Inc., Irvine, Calif. Voluma is especially desirable for injecting into the eye since it expands considerably less than the others and keeps the particles of anti-neovascular agent together thereby controlling release of the anti-neovacular agent in a sustained release manner.

The higher degrees of HA cross-linking, i.e. 4 to 8% and higher, makes the HA complex paradoxically hydrophobic in a highly aqueous media such as the vitreous, and surprisingly, there is reduced polymer expansion potential. In addition, reduced expansion makes the hydrogel more effective at “caging” the anti-neovascular agent drug particles and/or microspheres which in turn increases the duration of drug release in the vitreous.

15 Optionally, to increase the duration of release of the anti-neovascular agent monoclonal antibody from the viscous formulation, the antibody can be incorporated within microspheres which are and then formulated into the cross-linked HA. The microspheres can be simply mixed with the HA, or since the HA polymer is a polyanionic polysaccharide, a positive charge can be applied to the microspheres to create an electrostatic bond with the surrounding HA polymers. Variations in the HA concentration, molecular weight, and degree of cross-linking can be carried out in the formulation to influence particle agglomeration and containment within the drug depot.

The microsphere, can be composed of, but are not limited to, one or more of the following polymers: poly(lactide-co-glycolide), poly(DL-lactide-co-glycolide), poly(DL-lactide), poly(L-lactide-co-glycolide), polycaprolactone, poly(DL-lactide-co-caprolactone), poly(L-lactide-co-caprolactone), polyglycolide, and polylactide.

The microsphere/HA combination provides a 2-step drug release platform: a) HA allows the ingress of surrounding aqueous fluids into the depot which hydrates the microspheres and controlled drug release from the polymers, b) drug release from the surrounding HA polymers. Depending on the polymers used in the microspheres and the drug load, the formulation can release the drug for up to 6 months. The invention can be injected sub-Tenon's, or optionally, directly into the anterior chamber to treat diseases associated with corneal neovascularization.

Other advantages of our microsphere/HA combination formulations include:
1. after in vivo, intraocular injection our formulation shows the characteristics of rapid microsphere agglomeration in the HA vehicle which can increase the in vivo half-life of the drug depot.
2. encapsulation of the anti-neovascular agent with the polymers which constitute in the microspheres can protect the labile anti-neovascular agent protein.
3. the formulation can be injected using pre-filled syringes in which the microspheres are suspended.
4. the potential for hypodermic needle occlusion due to microsphere clumping during the injection is reduced because of the enhanced lubrication provided by the HA

Corneal neovascularization is a sequel of several inflammatory diseases of the anterior segment, such as infections, degenerative and traumatic disorders, extended contact lens wear, dry eye with or without filamentary keratitis, progressive corneal vascularization caused by graft-versus-host disease, limbal stem cell deficiency (including idiopathic, traumatic, aniridia, autoimmune polyendocrinopathy), Stevens-Johnson syndrome, ocular pemphigoid, HSV keratitis, and recurrent pterygium following surgery. Corneal graft rejection and failure is problematic in most patients with high risk characteristics. Among a host of factors predisposing to immune graft rejection of the corneal graft, vascularization of the host cornea prevails as the most important factor. Deep stromal vascularization of the host cornea of two or more quadrants classifies as a high-risk cornea. A previously rejected graft also serves as a significant predisposition to graft rejection as it pre-sensitizes the host, leading to a mounted immune response. Further, repeat corneal grafts are always associated with a lower chance of survival than the first graft. Young patients and bilateral graft have more chances of graft rejection due to active immune system. Neutralization of all VEGF isomers with the disclosed invention after high-risk corneal transplantation may effectively inhibit postoperative lymphangiogenesis, hemangiogenesis, and recruitment of antigen-presenting cells. Blocking this cascade of events and transport of donor tissue antigens into the regional lymph nodes would reduce the chance of corneal graft rejection. Furthermore, chronic exposure with anti-VEGF blockade may lead to apoptosis of endothelial cells and regression of pre-existing vessels in the host bed.

Other diseases that can be potentially treated with the invention are neovascular glaucoma and tumors of the anterior segment such as a ciliary body or iris melanoma. The invention can also include releasing anti-glaucoma and anti-ocular hypertension drugs for treating open angle glaucoma. In addition, posterior segment diseases including diabetic macular edema and age-related macular degeneration can also be treated with the invention.

Other anti-VEGF compounds can be used in place of an anti-VEGF monoclonal antibody (e.g. bevacizumab) in the invention and these include anti-VEGF aptamers (e.g. Pegaptanib), soluble recombinant decoy receptors (e.g. VEGF Trap), antibody fragments (e.g. Ranibizumab), corticosteroids, angiostatic steroids, anecortave acetate, angiostatin, endostatin, small interfering RNA's decreasing expression of VEGFR or VEGF ligand, post-VEGFR blockade with tyrosine kinase inhibitors, MMP inhibitors, IGFBP3, SDF-1 blockers, PEDF, gamma-secretase, Delta-like ligand 4, integrin antagonists, HIF-1 alpha blockade, protein kinase CK2 blockade, and inhibition of stem cell (i.e. endothelial progenitor cell) homing to the site of neovascularization using vascular endothelial cadherin (CD-144) and stromal derived factor (SDF)-1 antibodies. Small molecule RTK inhibitors targeting VEGF receptors including PTK787 can also be used. Agents that have activity against neovascularization that are not necessarily anti-VEGF compounds can also be used and include anti-inflammatory drugs, rapamycin, cyclosporine, anti-TNF agents, anti-complement agents, and nonsteroidal anti-inflammatory agents. Agents that are neuroprotective and can potentially reduce the progression of dry macular degeneration can also be used, such as the class of drugs called the ‘neurosteroids.’ These include drugs such as dehydroepiandrosterone(DHEA)(Brand names: Prastera® and Fidelin®), dehydroepiandrosterone sulfate, and pregnenolone sulfate.

Penetration enhancers can be used to increase the permeability of the tissues from the injected drug depot to the cornea. A preferred penetrant enhancer is polysorbate 20 (includes Tween 20, C12-sorbitan-E20) and polysorbate 80 in concentrations ranging from 0.005% to 0.10%. In addition, benzalkonium chloride is also a valuable agent that can increase transscleral drug delivery and increase drug levels in the anterior chamber.

The present invention is based upon our discovery of anti-neovascular agent-containing formulations specifically designed for intraocular, for example intracorneal, injection or administration to treat various ocular conditions, such a corneal neovascularization. Our anti-neovascular agent formulations have numerous superior characteristics and advantages, including the following: (1) our formulations may be made to be free of preservatives and resuspension aids, such as benzyl alcohol and/or a polysorbate; (2) concomitantly, our formulations have a much reduced retinal and photoreceptor toxicity; (3) as well as being sterile and optionally preservative-free, our anti-neovascular agent formulations can provide extended therapeutic effects due to the viscosity of the formulation and the relatively slow diffusion of the anti-neovascular agent there from, and when formulated as a suspension of particles, can provide sustained release of therapeutic amounts of the anti-neovascular agent over, for example, a period of months periods upon intravitreal injection of such formulations. Thus, our viscous anti-neovascular agent formulations can be characterized as sustained release implants; (4) intravitreal administration of our anti- neovascular agent formulations is substantially unassociated with an increased incidence of adverse events such as substantially elevated intraocular pressure, glaucoma, cataract and/an intraocular inflammation; (5) intravitreal administration of our anti-neovascular agent formulations is not associated with an increased incidence of adverse events such elevated intraocular pressure, glaucoma, cataract and/an intraocular inflammation as compared to currently used or known intraocular (e.g., intravitreal) use anti-neovascular agent formulations; (6) in certain embodiments, our formulations permit anti-neovascular agent particles or crystals to be slowly released (as they solubilize in the viscous fluid of the posterior chamber) from a relatively discrete unitary location, thereby avoiding the plume effect (rapid dispersion) characteristic of less viscous aqueous formulations upon intravitreal administration; (7) avoidance of plume formation or rapid dispersion upon intravitreal administration, which beneficially reduces visual field obscuration.

Advantage (3) above can be provided by particular characteristics of our formulations, such as suspension of the anti-neovascular agent in one or more particular high molecular weight polymers which permit sustained release of the anti-neovascular agent by the formation of ion pairing or reverse phase association therewith. Thus, the anti-neovascular agent is slowly related from its association with the gel.

Depending on the solubility of the anti-neovascular agent, the anti-neovascular agent can be present in the present compositions in an amount in the range of about 1% or less to about 5% or about 10% or about 20% or about 30% or more (w/v) of the composition, or about 0.2 mg per 100 μl or about 0.4 mg per 100 μl, or about 0.5 mg per 100 μl, or about 1.0 mg per 100 μl or about 2.0 mg per 100 μl, or about 4.0 mg per 100 μl, or about 5.0 mg per 100 μl, or about 6.0 mg per 100 μl, or about 7.0 mg per 100 μl, or about 8.0 mg per 100 μl, or about 10 mg per 100 μl, or about 20 mg per 100 μl, or about 40 mg per 100 μl, or about 60 mg per 100 μl, or about 80 mg per 100 μl. Providing relatively high concentrations or amounts of anti-neovascular agent in the present compositions is beneficial in that reduced volumes and frequency of dosages of the composition may be required to be placed or injected into the posterior segment of the eye in order to provide the same amount or more anti-neovascular agent in the posterior segment of the eye relative to compositions which include less than about 4% (w/v) of the anti-neovascular agent. Thus, in one very useful embodiment, the present compositions include more than about 4% (w/v), for example at least about 5% (w/v), to about 10% (w/v) or about 20% (w/v) or about 30% (w/v) of the anti-neovascular agent. Intraocular injection of 100 μL or more of a fluid can result in an excess of fluid with elevated intraocular pressure and leakage of the fluid from the intraocular site then potentially occurring.

The polymeric hyaluronic acids present in an effective amount in increasing, advantageously substantially increasing, the viscosity of the composition. Without wishing to limit the invention to any particular theory of operation, it is believed that increasing the viscosity of the compositions to values well in excess of the viscosity of water, for example, at least about 100 cps at a shear rate of 0.1/second, compositions which are highly effective for placement, e.g., injection, into the posterior segment of an eye of a human or animal are obtained. Along with the advantageous placement or injectability of the present compositions into the posterior segment, the relatively high viscosity of the present compositions are believed to enhance the ability of the present compositions to maintain the anti-neovascular agent localized for a period of time within the posterior segment after intravitreal injection or placement. In the event that the composition comprises particles or crystals of the anti-neovascular agent, the viscosity of the composition maintains the particles in substantially uniform suspension for prolonged periods of time, for example, for as long as 1 to 2 years, without requiring resuspension processing and thereby increasing the effective shelf life of the composition. The relatively high viscosity of the present compositions may also have an additional benefit of at least assisting the compositions to have the ability to have an increased amount or concentration of the anti-neovascular agent, as discussed elsewhere herein.

Advantageously, the present compositions have viscosities of at least about 10 cps or at least about 100 cps or at least about 1000 cps, more preferably at least about 10,000 cps and still more preferably at least about 70,000 cps or more, for example up to about 200,000 cps or about 250,000 cps, or about 300,000 cps or more, at a shear rate of 0.1/second. The present compositions not only have the relatively high viscosity as noted above but also have the ability or are structured or formed to be effectively placeable, e.g., injectable, into a posterior segment of an eye of a human or animal, preferably through a 27 gauge needle, or even through a 30 gauge needle.

The presently useful polymeric hyaluronic acid preferably are shear thinning components in that as the present composition containing such a shear thinning polymeric hyaluronic acid is passed or injected into the posterior segment of an eye, for example, through a narrow space, such as 27 gauge needle, under high shear conditions the viscosity of the composition is substantially reduced during such passage. After such passage, the composition regains substantially its pre-injection viscosity.

Any suitable viscosity inducing component, for example, ophthalmically acceptable viscosity inducing component, may be employed in accordance with the present invention. For example a polysaccharide can be used in ophthalmic compositions used on or in the eye. Preferably, compositions for treating ocular neovasculization within the scope of the present invention comprise a polysaccharide which is a hyaluronic acid. More preferably, the hyaluronic acid used in the composition is a hyaluronic acid polymer which is present in an amount effective to provide a desired viscosity to the composition. Advantageously, (and depending on its properties and average molecular weight) the hyaluronic acid polymer is present in an amount in a range of about 0.5% or about 1.0% to about 5% or about 10% or about 20% (w/v) of the composition. The specific amount of the hyaluronic acid polymer employed depends upon a number of factors including, for example and without limitation, the synthesis route of the specific hyaluronic acid polymer being employed, the molecular weight of the hyaluronic acid polymer used the viscosity desired for the composition and similar factors, such as shear thinning, biocompatibility and possible biodegradability of the compositions.

A composition within the scope of our invention preferably comprises a hyaluronic acid polymer to provide several desirable characteristics to the composition, such as to increase the viscosity of the composition, to provide a sustained release of the anti-neovascular agent from the composition and to provide a composition with a low intraocular immunogenicity.

Examples of useful polymers include, but are not limited to, a hyaluronic acid polymer, carbomers, polyacrylic acid, cellulosic derivatives, polycarbophil, polyvinylpyrrolidone, gelatin, dextrin, polysaccharides, polyacrylamide, polyvinyl alcohol, polyvinyl acetate, derivatives thereof and mixtures and copolymers thereof. In a particularly preferred embodiment the composition comprises a hyaluronic acid component, such as a hyaluronic acid polymer component, including a cross-linked hyaluronic acid polymer.

An average molecular weight of the presently useful hyaluronic acid polymer can be in a range of about 10,000 Daltons or less to about 2 million Daltons or more. In one particularly useful embodiment, the molecular weight of the hyaluronic acid polymers is in a range of about 100,000 Daltons or about 200,000 Daltons to about 1 million Daltons or about 1.8 million Daltons. Again, the molecular weight of the hyaluronic acid polymer is useful in accordance with the present invention, may vary over a substantial range based on the type of hyaluronic acid polymers employed, and the desired final viscosity of the present composition in question, as well as, possibly one or more other factors. In one embodiment, two or more distinct molecular weight ranges of the hyaluronic acid polymers may be used to increase the shear thinning attributes of the composition.

In one very useful embodiment, a hyaluronic acid polymer acid is for example, a polymeric, metal hyaluronate component, preferably selected from alkali metal hyaluronates, alkaline earth metal hyaluronates and mixtures thereof, and still more preferably selected from sodium or potassium hyaluronates, and mixtures thereof. The molecular weight of such hyaluronate component (i.e. a hyaluronic acid polymer) preferably is in a range of about 50,000 Daltons or about 100,000 Daltons to about 1.3 million Daltons or about 2 million Daltons. In one embodiment, the present compositions include a polymeric hyaluronate component in an amount in a range about 0.05% to about 0.5% (w/v). In a further useful embodiment, the hyaluronate component is present in an amount in a range of about 1% to about 4% (w/v) of the composition. In this latter case, the very high polymer viscosity forms a gel that slows particle sedimentation and diffusion of dissolved solutes upon injection in the eye. Such a composition may be marketed in pre-filled syringes since the gel cannot be easily removed by a needle and syringe from a bulk container. Pre-filled syringes have the advantages of convenience for the injector and the safety which results from less handling and the opportunity for error or contamination.

The present compositions preferably include at least one buffer component in an amount effective to control and/or maintain the pH of the composition and/or at least one tonicity component in an amount effective to control the tonicity or osmolality of the compositions; preferably the tonicity and/or osmolality will be substantially isotonic to the vitreous humor. More preferably, the present compositions include both a buffer component and a tonicity component.

The buffer component and tonicity component may be chosen from those which are conventional and well known in the ophthalmic art. Examples of such buffer components include, but are not limited to, acetate buffers, citrate buffers, phosphate buffers, borate buffers and the like and mixtures thereof. Phosphate buffers are particularly useful. Useful tonicity components include, but are not limited to, salts, particularly sodium chloride, potassium chloride, mannitol and other sugar alcohols, and other suitable ophthalmically acceptably tonicity component and mixtures thereof.

The amount of buffer component employed preferably is sufficient to maintain the pH of the composition in a range of about 6 to about 8, more preferably about 7 to about 7.5. The amount of tonicity component employed preferably is sufficient to provide an osmolality to the present compositions in a range of about 200 to about 400, more preferably about 250 to about 350, mOsmol/kg respectively.

Advantageously, the present compositions are substantially isotonic. The present compositions may include one or more other components in amounts effective to provide one or more useful properties and/or benefits to the present compositions. For example, although the present compositions may be substantially free of added preservative components, in other embodiments, the present compositions include effective amounts of preservative components, preferably such components which are more compatible with the tissue in the posterior segment of the eye into which the composition is placed than benzyl alcohol. Examples of such preservative components include, without limitation, benzalkonium chloride, chlorhexidine, PHMB (polyhexamethylene biguanide), methyl and ethyl parabens, hexetidine, chlorite components, such as stabilized chlorine dioxide, metal chlorites and the like, other ophthalmically acceptable preservatives and the like and mixtures thereof. The concentration of the preservative component, if any, in the present compositions is a concentration effective to preserve the composition, and is often in a range of about 0.00001% to about 0.05% or about 0.1% (w/v) of the composition.

Solubility of the anti-neovascular agent is clearly important to the effectiveness of the present anti-neovascular agent-containing compositions, as is the potency and efficacy of the anti-neovascular agents themselves. Very soluble anti-neovascular agents are more readily and immediately available to the intraocular tissues, but may accordingly require smaller doses of the anti-neovascular agent (and more frequent administration) to avoid substantially exceeding the effective dose. The viscosity of the present compositions will, to some extent, slow the diffusion of even these very soluble anti-neovascular agents, but will not as effectively provide for an extended period of delivery and resulting efficacy as, for example is true when the anti-neovascular agent is sequestered or somewhat insoluble (and thus solubilized over a period of time in situ) in the anti-neovascular agent composition of the present invention. The availability of minimally soluble anti-neovascular agents to intraocular tissues may be limited by the dissolution rate for these substances. As with readily soluble anti-neovascular agents, slow dissolution is both good and bad for the patient. On the one hand, after a single intravitreal injection of the present composition, the mean elimination half-life for the anti-neovascular agent is advantageously quite long. On the other hand, therapeutic drug levels in the vitreous compartment of the eye may not be achieved for some time (for example, about 1 to about 3 days), due to the slow dissolution rate of the anti-neovascular agent particles.

In one embodiment of the present invention, the compositions further contain sustained release components, for example, polymers (in the form for example of gels and microspheres), such as poly (D,L,-lactide) or poly(D,L-lactide co-glycolide), in amounts effective to reduce local diffusion rates and/or anti-neovascular agent particle dissolution rates. The result is a flatter elimination rate profile with a lower Cₘₐₓ and a more prolonged therapeutic window, thereby extending the time between required injections for many patients.

Any suitable, preferably conditionally acceptable, release component may be employed. Useful examples are set forth above. The sustained release component is preferably biodegradable or bioabsorbable in the eye so that no residue remains over the long term. The amount of the delayed release component included may very over a relatively wide range depending, for example, on the specific sustained release component is being employed, the specific release profile desired and the like factors. Typical amounts of delayed release components, if any, included in the present compositions are in a range of about 0.05 to 0.1 to about 0.5 or about 1 or more percent (w/v) (weight of the ingredient in the total volume of the composition) of the composition.

The present compositions can be prepared using suitable blending/processing techniques or techniques, for example, one or more conventional blending techniques. The preparation processing should be chosen to provide the present compositions in forms which are useful for placement or injection into the posterior segments of eyes of humans or animals. Soluble anti-neovascular agent can be simply mixed with a hyaluronic acid solution. In one useful embodiment utilizing a somewhat insoluble anti-neovascular agent, a anti-neovascular agent dispersion is made by combining the anti-neovascular agent with water, and the excipient (other than the viscosity inducing component) to be included in the final composition. The ingredients are mixed to disperse the anti-neovascular agent and then autoclaved. Alternatively, the anti-neovascular agent particles may be γ-irradiated before addition to the sterile carrier. The polymeric hyaluronic acid_may be purchased sterile or sterilized by conventional processing, for example, by filtering a dilute solution followed by lyophylization to yield a sterile powder. The sterile polymeric hyaluronic acid_is combined with water to make an aqueous concentrate. Under aseptic conditions, the concentrated anti-neovascular agent dispersion can be blended or mixed and added or combined as a slurry to the polymeric hyaluronic acid_concentrate. Water is added in a quantity sufficient (q.s.) to provide the desired composition and the composition is mixed until homogenous.

Methods of using the present composition are provided and are included within the scope of the present invention. In general, such methods comprise administering a composition in accordance with the present invention to a posterior segment of an eye of a human or animal, thereby obtaining a desired therapeutic effect, such as treatment of a given condition of the anterior or posterior segment of the eye. The administering step advantageously comprises at least one of intravitreal injecting, subconjunctival injecting, sub-tenon injecting, retrobulbar injecting, suprachoroidal injecting and the like. A syringe apparatus including an appropriately sized needle, for example, a 27 gauge needle or a 30 gauge needle, can be effectively used to inject the composition with the posterior segment of an eye of a human or animal.

Ocular conditions which can be treated or addressed in accordance with the present invention include, without limitation, the following:

Maculopathies/retinal degeneration: macular degeneration, including age related macular degeneration (ARMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease. Traumatic/surgical: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like.

### EXAMPLES

The following non-limiting Examples are presented to exemplify aspects of the present invention.

### Example 1

### Rapid Drug Clearance from Sub-Tenon Space

We hypothesized that the lymphatic system and blood vessels present within the conjunctiva and sclera is able to eliminate small and large molecular weight drugs (such as anti-VEGF monoclonal antibodies) from an intraocular (i.e. intra-scleral, such as sub-tenon) site to which a low viscosity, aqueous drug solution is administered. Elimination being to the regional lymph nodes and thence out of the eye.

We had previously determined that PLGA microspheres (not in a high viscosity vehicle) injected in the sub-Tenon's space cleared rapidly (within six hours) out of the sub-tenon's space, thereby limiting the value of such microspheres to treat an ocular disease.

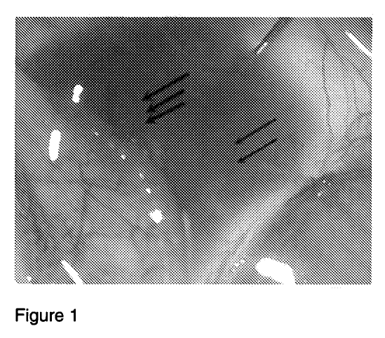

Thus, to evaluate the clearance mechanisms of an aqueous solution a tracer dye was injected in the sub-Tenon's space in a rabbit eye and the time to disappearance of the dye was determined as follows. A 2-3 kg New Zealand Rabbit was given general anesthesia. The right eye was retracted inferiorly with a 9-0 vicryl suture through the cornea. In the superotemporal quadrant, 100 μl of Alexa fluor 647 dye (Invitrogen, Carlsbad, Calif.) was injected in the sub-Tenon's space using a 30 G hypodermic needle (see FIG. 1). Serial examinations showed that the Alexa dye was cleared completely from the sub-Tenon's space within 6 hours.

### Example 2

### Intraocular Durability of Cross Linked Hvaluronic Acid in Sub-Tenon Space

An experiment was carried out demonstrating that a cross-linked, polymeric hyaluronic acid has long-term durability and tolerability in the sub-Tenon's space and therefore suitability to act as a drug carrier for sustained drug delivery.

A 300 gram Sprague-Dawley Rat was placed under general anesthesia and one eye was injected in the sub-Tenon's space with 10 μl of the polymeric hyaluronic acid formulation Juvederm Ultra Plus (Allergan, Irvine, Calif.). The polymeric hyaluronic acid can be used at a concentration of about 20 mg/ml. An alternate polymeric hyaluronic acid which can be used comprises about 95% crosslinked hyaluronic acid and 5% uncrosslinked (free-flowing) hyaluronic acid. The uncrosslinked hyaluronic acid can have an average molecular weight between 600-1,500 kDa, and the cross linked HA component can have an average molecular weight of greater than about 400 kDa. Magnetic resonance imaging with a 7 Tesla Bruker MRI PharmScan was performed to directly visualize the cross-linked HA without contrast.

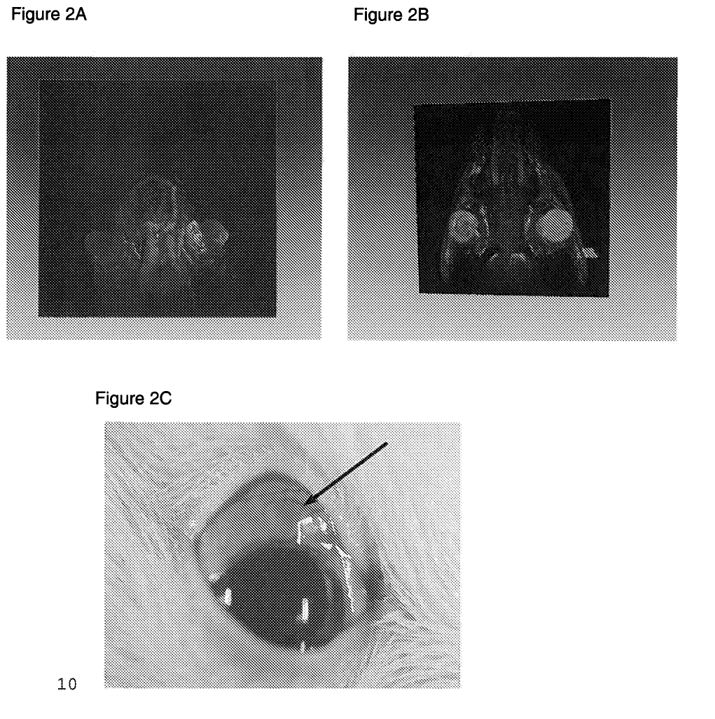

The volume of HA injected sub-tenon on Day 0 (at +55 minutes) was 24.41 cubic millimeters (see FIG. 2A). The volume of the HA remaining sub-tenon at Day 90 after injection was 7.18 cubic millimeters or 30% of the initial injection volume (see FIG. 2B). Quantitative analysis using iMura imaging software was used to determine the HA volume at day 90.

Thus, despite lymphatic presence of lymphatic elimination mechanisms about one-third of the injected polymer HA remained intraocular for at least 3 months. Hence, cross-linked HA with its long term durability in the sub-Tenon's space can be used as the vehicle for an active agent in a sustained release drug delivery system. Clinical examination of the eye after 3 months demonstrated that the polymer used in this invention is well-tolerated and there were no signs of any ocular pathology (see FIG. 2C).

FIG. 2A is an MRI showing cross-linked hyaluronic acid (HA) in the sub-Tenon's space 55 minutes following an injection in a rat eye. The polymer is highlighted in purple for quantification. The +55 minute scan was a FISP-3D scan made with these parameters: FOV (field of view) 4.5 cm, Matrix dimensions 256×256×256 (176 micron resolution), TR 8 ms, TE 4 ms, 16 echoes, 4 averages, slice thickness (ST) 45 mm, coronal, Time 20 m 28 s

FIG. 2B follow-up MRI 3 months after injection of a cross-linked HA (purple). The +3 month scan was a MSME-T2-map scan (multi-spin multi-echo) made with these parameters: FOV (field of view) 4 cm, Matrix dimensions 256×256 (156 micron resolution), TR 1725.6 ms, TE 10 ms, 16 echoes, 2 averages, slice thickness (ST) 0.75 mm, inter-slice distance (ISD) 1 mm, 10 slices, coronal, Time 11 m 2 s FIG. 2C is a clinical photograph of the rat eye 3 months post-injection with a cross-linked HA. The arrow points to a depot which shows intraocular presence of the HA.

### Example 3

### Intraocular Durability of Microspheres in Hyaluronic Acid in Sub-Tenon Space

An experiment was carried out demonstrating that a polymeric hyaluronic acid (HA) can be used as a vehicle for and can retain microspheres in an intraocular depot formulation over a period of at least a 1 month period.

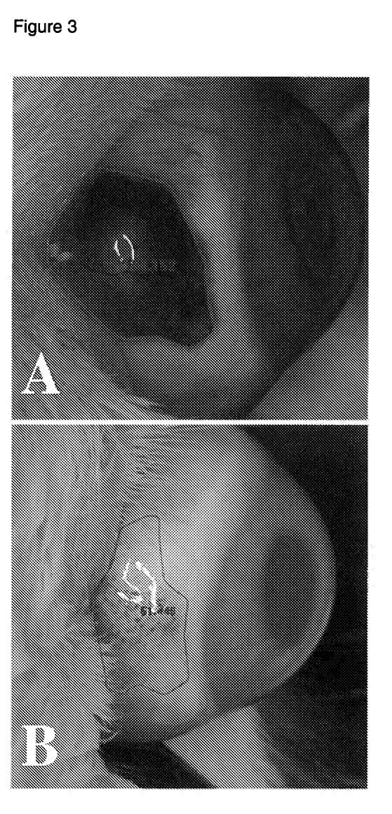

Thus, a cross linked, polymeric HA (the same HA used in Example 2, that is Juvederm Ultra Plus) was used to investigate its ability to retain surrogate drug microspheres in following injection into the sub-Tenon's space. The experiment was carried out as follows. A 2-3 kg New Zealand Rabbit was given general anesthesia. Colored microspheres were used as surrogate for similar sized microspheres that are used clinically for drug delivery. The microspheres used were “Dye-trak microspheres” with an average diameter of 15 microns, obtained from Triton Technology Inc. as part number 145-0672. The right eye of the rabbit was injected with 15 μm diameter Dye-Trak Microspheres (Triton Tech, Part#1450672 Blue, Lot#15TB, 30 million in 10 ml) into the sub-Tenon's space, superotemporally (see FIG. 3A). The total surface area of the depot 55 minutes after sub-tenon injection was 54.152 mm². One month after the sub-tenon injection the total surface area of the depot was 51.446 mm², and some microspheres were visually present in the polymer (see FIG. 3B), amounting to a 5% reduction in surface area after one month, as shown by the FIGS. 3A and FIG. 3B photographs. This shows that the HA polymer used has the ability to deliver drug for a prolonged period of time since the HA polymer is present for a long duration in the sub-Tenon's space.

FIG. 3A is a clinical photograph of a rabbit eye immediately following a sub-Tenon's injection microspheres in a cross-linked HA. The red line area outlines the periphery of the drug depot and the surface area is 54.152 mm^{2.}

FIG. 3B is a clinical photograph 1 month after injection of the microspheres in a cross-linked HA injected in FIG. 3A. Note that some microspheres are still present in the depot after 1 month. The red line area outlines the periphery of the drug depot and the surface area is 51.446 mm²

### Example 4

### Treatment of Corneal Neovascularization with a Bevacizumab-HA Formulation

A 57 year old man has a history of an occupational chemical injury to the right eye 5 years previously and his visual acuity in the right eye is 20/400. Slit-lamp examination can reveal 3-quadrant corneal neovascularization with vessels extending to the center of the cornea. There is a deep stromal scar centrally in the right eye. The patient is seen by a corneal specialist for consideration of a penetrating keratoplasty (PKP) but is told he is high-risk for a rejection and possible loss of the graft because of the corneal neovascularization. The patient can decide to proceed with the PKP and do well with a clear graft for about 2 months with visual acuity improvement to 20/100. The patient can complain 2 months later of reduced vision and a red eye. The patient is seen immediately in the office and is diagnosed with an endothelial immune rejection. Despite aggressive topical and sub-Tenon's corticosteroids to abort the rejection response, the cornea can develop stromal edema and the patient's visual acuity can return to pre-PKP levels. The patient can now have 4-quadrant neovascularization of the graft bed. One year can pass since the loss of the initial graft and the patient is considering a repeat PKP. One month prior to the repeat PKP he can receive a sub-Tenon's injection of bevacizumab in crosslinked or uncrosslinked hyaluronic acid polymer or a combination of the two. The bevacizumab can be used at a concentration of 1.25 mg/50 microliter and the formulation is injected into the anterior sub-Tenon's. Total volume of the formulation injected can be 100 microliter. The patient can then have clear regression of the corneal vessels at the 2 week post-injection visit. At 2 months there can be significant vessel regression and they can appear dormant. The patient can undergo a repeat PKP and at 6 months post-operatively there can be no episodes of graft rejection and the cornea can be clear. The visual acuity can have improved to 20/60.

### Example 5

### Treatment of Corneal Neovascularization with a Ranibizumab-HA Formulation

A 19 year old woman develops generalized tonic-clonic seizures at age 14 years and after an extensive neurologic work-up can be placed on phenytoin (Dilantin). Six weeks after starting the medication she can develop Stevens-Johnson syndrome (erythema multiforme major) with ocular involvement. The patient can develop significant conjunctival scarring and corneal neovascularization in both eyes with vision loss to 20/400. Given that the patient is at high risk for a corneal graft failure, she can undergo a limbal stem cell transplant (allograft) in the right eye followed by a penetrating keratoplasty 6 months later. She is also given a sub-Tenon's injection of a therapeutic composition within the scope of our invention which in this embodiment is a thick gel containing crosslinked or uncrosslinked hyaluronic acid polymer or a combination of the two with a total of 1 mg of ranibizumab in a 100 ul total volume. The patient's clinical course goes well and there are no grafts rejection episodes. Furthermore, the pre-existing vessels in the graft bed are now ghost vessels and perfusion with RBCs (red blood cells) is no longer visible by slit-lamp.

### Example 6

### Treatment of Iris Neovascularization with a Bevacizumab-HA Formulation

A 68 year old woman complains of blurry vision in her left eye and was seen by her general ophthalmologist. She has visual acuity of CF 3 ft left eye with an ischemic central retinal vein occlusion with numerous cotton wool spots apparent in the posterior pole. The patient is watched closely and develops neovascularization of the iris 3 months following the vein occlusion. The intraocular pressure (IOP) increases to 42 mmHg and the angle can show fine new vessels coursing through the trebecular meshwork with anterior synechiae noted temporally. The patient can receive a subTenon's injection of a therapeutic composition within the scope of our invention which in this embodiment is a thick gel containing crosslinked or uncrosslinked hyaluronic acid polymer or a combination of the two with a total of 2.5 mg of bevacizumab in the injected volume of 100 ul. After 2 weeks, the IOP can be 26 mmHg with the iris neovascularization improved.

Advantages of our invention (for example the discovery that cross linked HA can be used as a carrier to increase intraocular anti-neoplastic agent residency time at the site (i.e. sub-tenon or intravitreal) of administration) include: sustained release in vivo of an anti-neovascular agent over a period of time of up to six months; reduces the need for monthly injections to treat CNV (choroidal neovascularization), and provides a prophylaxis therapy for CNV in high risk eyes

### Example 7

### Process for Making Therapeutic Composition

The therapeutic compositions set forth in Example 4-6 can be made using a process for making a composition for treating ocular neovascularization, the composition comprising an anti-neovascular agent, and a polymeric hyaluronic acid associated with the anti-neovascular agent, wherein the polymeric hyaluronic acid is present in the composition at a concentration between about 10 mg/ml and about 30 mg/ml. The process can comprise the following steps:
(a) solubilize and stabilize the anti-neovascularization agent in solution containing a stabilizing agent (such as eg threhaloses, sucrose, maltose, polyethylene glycol, polysorbate 20, tranexamic acid, aminocaproic acid, L-lysine, and analogs of L-lysine, L-arginine, L-ornithine, aminobutyric acid, glycylglycine, gelatin, albumin and glycerin) a buffer (eg an acetate, citrate, phosphate or borate buffer) and/or an isotonizing salt. Sterile filter the solution through a 0.2 micron filter.
(b) lyophilize the drug solution so that the result is a dry powder cake; The lyophilization cycle can be as follows:

1) Decrease temperature to 5 C at 2 C/minute. Hold for 30 minutes

2) Decrease temperature to −45 C at 1 C/minute. Hold for 120 minutes

3) Final freeze at −45 C for 60 minutes at 100 mTorr

4) Hold at −45 C and 100 mTorr for 1800 minutes

5) Ramp up to 0 C at 0.1 C/minute. Hold for 300 minutes at 100 mTorr

6) Ramp to 5 C at 0.1 C/min. Hold for 300 minutes at 100 mTorr

7) Ramp to 20 C at 0.1 C/min. Hold for 300 minutes at 100 mTorr

8) Ramp to 25 C at 0.1 C/min. Hold for 1440 minutes at 100 mTorr

9) Back-fill with nitrogen to reach atmospheric pressure
(c) Under clean or sterile conditions, blend the powder and the hyaluronic acid polymer(s) using an overhead mixer, spatula, shear forces, vortex mixer, ball mill, other means, or by coupling two syringes together with the lyophilized powder in one syringe the gel in the other syringe and mixing back and forth between the two chambers. Transfer the gel formulation to luer-lock capped syringes or conical vials.
d) centrifuge the composition at a range of 1500-5000 RPM for 5 to 60 minutes to evacuate air from the gel.
e) Transfer gel to sterile syringes using a clean stainless steel spatula, suction, or a luer-lock to luer-lock connector. Centrifuge the syringes if necessary to evacuate air from gel.

All patents, patent applications and publications cited herein are hereby incorporated by reference in their entireties. The invention is set forth by the following claims, the spirit and scope of which is not intended to be limited to the examples and embodiments set forth above.

We claim:

1.-13. (canceled)

14. A method for treating ocular neovascularization, the method comprising the step of administering to the eye of patient exhibiting ocular neovascularization a therapeutic amount of a composition comprising an anti-neovascular agent, and a polymeric hyaluronic acid associated with the anti-neovascular agent, wherein the polymeric hyaluronic acid is present in the composition at a concentration between about 10 mg/ml and about 30 mg/ml.

15. The method of claim 14, wherein the ocular neovascularization is corneal neovasculization.

16. The method of claim 15, wherein the anti-neovascular agent is bevacizumab.

17. A method for treating corneal neovascularization, the method comprising the step of administering to the eye of patient exhibiting corneal neovascularization a therapeutic amount of a composition comprising bevacizumab, and a polymeric hyaluronic acid associated with the bevacizumab, wherein the polymeric hyaluronic acid is present in the composition at a concentration between about 10 mg/ml and about 30 mg/ml.

18. (canceled)

19. The method of claim 14, wherein the polymeric hyaluronic acid is present in the composition at a concentration between about 20 mg/ml and about 30 mg/ml.

20. The method of claim 14, wherein the polymeric hyaluronic acid comprises from about 1 weight % to about 50 weight % cross-linked polymeric hyaluronic acid.

21. The method of claim 20, wherein the polymeric hyaluronic acid comprises from about 1 weight % to about 10 weight % cross-linked polymeric hyaluronic acid.

22. The method of claim 20, wherein the cross-linked, polymeric hyaluronic acid, is made from non-cross linked polymeric hyaluronic acid which has a molecular weight between about 200 kDa and about 2,000 kDa.

23. The method of claim 14, wherein the hyaluronic acid has a storage modulus (G′) of between about 200 and 400 at 5 Hz at 25° C.

24. The method of claim 14, wherein the composition further comprising biodegradable, polymeric microspheres.

25. The composition of claim 12, wherein the microspheres incorporate at least some of the anti-neovascular agent.
